(19) United States
(12) Patent Application Publication
(10) Pub. No.: US 2006/0127466 A1
(43) Pub. Date: Jun. 15, 2006

(54) Method for transdermal or intradermal delivery of molecules

(76) Inventors: Arindam Sen, Williamsville, NY (US); Sek Wen Hui, Williamsville, NY (US); Ya Li Zhao, Amherst, NY (US); Lei Zhang, San Diego, CA (US)

Correspondence Address: HODGSON RUSS LLP, ONE M & T PLAZA, SUITE 2000, BUFFALO, NY, 14203-2391, US

(21) Appl. No.: 11/263,223

(22) Filed: Oct. 31, 2005

Related U.S. Application Data
(63) Continuation-in-part of application No. 09/793,256, filed on Feb. 26, 2001, now abandoned.
(60) Provisional application No. 60/184,918, filed on Feb. 25, 2000.

(51) Int. Cl. A61N 1/30; A61K 9/127

(52) U.S. Cl. 424/450; 604/20

(57) ABSTRACT

The present invention provides a method for transdermal delivery of molecules. The method comprises the application of electrical pulses concurrently or sequentially with application of the molecules and a lipid composition comprising negatively charged liposomal compositions. The application of the liposomal components enhances permeability of the target site for delivery of the molecule even though the molecules are not encapsulated in the liposomal components.

This application is a continuation-in-part of U.S. non-provisional patent application Ser. No. 09/793,256 filed on Feb. 26, 2001, which in turn claims priority to U.S. Provisional application Ser. No. 60/184,918 filed on Feb. 25, 2000.

### FIELD OF INVENTION

The present invention relates generally to the field of delivery systems for molecules. More particularly, the present invention provides a method for intradermal or transdermal delivery of molecules comprising electroporating the skin concurrently or sequentially in relation to the application of the molecules and a liposomal composition to the skin.

### DISCUSSION OF RELATED ART

Transdermal and intradermal drug delivery has many potential advantages over other delivery methods. Apart from the convenience and non-invasiveness, it offers a transport route that avoids degradation or metabolism of the introduced molecules by the gastrointestinal tract or liver. The skin also can provide a “reservoir” that sustains the delivery of introduced molecules over a period of days (Cullander, 1992, Advanced Drug Delivery Reviews 9:119-135). Furthermore, it offers multiple sites of delivery to avoid local irritation and toxicity, and it is possible to concentrate drugs at local areas to avoid undesirable systemic effects.

Topically applied drugs have many applications including treatments of osteoarthritis, soft-tissue rheumatism, tendinitis, local inflammatory conditions, cosmetic applications, and a variety of skin carcinomas, to name a few. The skin is also a site of vaccine delivery. However, at present, the clinical use of transdermal delivery is limited by the fact that very few drugs, agents, nucleic acids, or other chemicals can be transported transdermally at a pharmaceutically relevant rate. This is because the skin forms an efficient barrier for most molecules, and very few non-invasive methods are known to significantly facilitate the penetration of this barrier.

Mammalian skin has two layers, the epidermis and the dermis. The epidermis is a stratified squamous keratinizing epithelium. The uppermost stratum of the epidermis is the stratum corneum (SC) which consists of about twenty layers of flattened, enucleate, keratin-filled corneocytes surrounded by lamellae of about eight lipid bilayers on average. The bilayers consist primarily of cholesterol, free fatty acids and ceramide. The total thickness of the SC varies from 10 to 40 μm, with an average thickness of 20 μm (Chizmadzhev et al., 1995;Biophysical Journal, 68:749-765; Bouwstra et al., 1995, J. Lipid Res. 36:685-695; Swartzendruber et al., 1989, Journal of Investigative Dermatology, 92:251-257). This layer constitutes the major electric resistance of the skin, and is the main barrier to substance transport. The skin resistance Rₛ is typically 5-25 kOhm/cm², whereas the capacitance Cₛ is 1-20 nF/cm² (DeNuzzio and Berner, 1990, Journal of Controlled Release 11:105-112). The skin also contains various appendages such as hair follicles, apocrine and apoeccrine sweat glands, and in humans, eccrine sweat glands, all of which are highly vascularized. These appendages also provide routes for substance exchange with the outside environment (Scott et al., 1993, Pharmaceutical Research 10:1699-1709).

Most transdermal delivery to-date has been by passive diffusion through appendages, using skin patches, lotions and creams. Different approaches have been proposed to enhance delivery of chemicals transdermally. For example, iontophoresis has been proposed which uses a weak, long-lasting DC field to transport molecules through the SC via appendageal or paracellular space. The non-permeable nature of the SC has limited the use of diffusion and iontophoresis to delivering small molecules, e.g., less than about 400 Daltons, over rather long application times, e.g., about tens of minutes to days.

Another approach to transdermal introduction of molecules has been to transiently permeabilize a membrane or skin by the application of a single or multiple short duration pulses (e.g., microseconds to milliseconds). This causes a predominant voltage gradient to develop through a cell across the non-conductive plasma membrane and, likewise, the voltage gradient across the skin develops across the non-conductive SC. If the voltage gradient exceeds the barrier breakdown potential, pores are formed and may reseal depending on the applied pulse field and duration. During the lifetime of the pores, materials may be transported across the barrier. This process is generally termed electroporation. Another method for transdermal delivery is through the use of liposomes. Liposomes have been used for topical transdermal drug administration with varying degrees of effectiveness, and the mechanism is still debatable. When applied to the histocultured murine skin surface, neutral liposomes were reported to concentrate in the hair follicle channels (Li et al., 1992, In Vitro Cell Dev. Biol. 28A:679-681; Li et al., 1993, In Vitro Cell Dev. Biol. 29A:258-260). Liposomes containing phosphatidylcholine alone, or a mixture of phosphatidylcholine, phosphatidyl-ethanolamine and cholesterol, have been utilized to deliver plasmids containing the lacZ reporter gene, to transfect the follicular epithelium (Li et al., 1995, Nature Medicine I(7):705-706). Alexander et al., (1995, Human Molecular Genetics 4(12):2279-2285) reported applying liposomes containing the cationic lipid dioleoyl-trimethylammonium propane (DOTAP) complexed to a plasmid pIRV-neo-K5 to mouse skin surface, and found widespread transfection of dermal fibroblasts including interfollicular epidermis and hair follicles. In conjunction with an applied electric field, VutIa et al. (1996, Journal of Pharmaceutical Sciences 85(1): 5-8) measured the “iontophoretic” transport of enkephalin encapsulated in charged and neutral liposomes across dermatomed human skin using a Franz chamber. They found that the use of charged liposomes did not enhance the iontophoretic transport, but helped to stabilize the drug against degradation. Hofmann et al. (1995, Bioelectrochemistry & Bioenergetics 38:209-222), Zhang et al. (1996, Biochemical and Biophysical Research Communication 220:633-636) and U.S. Pat. Nos. 5,464,386, 5,688,233, 5,462,520 suggested using one or more electric pulses to deliver macromolecules encapsulated in vesicles or microspheres or mixed with particles through the SC. U.S. Pat. Nos. 5,464,386, 5,688,233, 5,462,520 are incorporated herein by reference.

Electroporation of substances, including drugs, chemicals, and nucleic acids, into and through SC and skin also is described in U.S. Pat. No. 5,318,514, U.S. Pat. No. 5,968,066, U.S. Pat. No. 6,009,345, U.S. Pat. No. 6,132,419, WO 00/09205, WO 00/02621, WO 00/02620, all of which are assigned to Genetronics, Inc., and all of which are incorporated herein by reference.

Despite advances that have been made, there is an ongoing need to develop methodologies for enhancing transdermal delivery of desired molecules.

### SUMMARY OF THE INVENTION

The present invention provides a method for transdermal and intradermal delivery of molecules. The method comprises the application of electrical pulses concurrently or sequentially with application of the molecules and a lipid composition comprising negatively charged liposomal compositions. The liposomal components are used to enhance permeability of the target site for delivery of the molecule. This invention can be used to facilitate the transport of molecules by electroporation, including large, neutral molecules that have previously been difficult to transport. In one embodiment of the invention, the liposomal composition is comprised of phospholipids including but not limited to dioleoylphosphatidylglycerol (DOPG) and dioleoylphosphatidylcholine (DOPC). The lipid compositions may, but need not be formed into liposomes or other structures to provide the enhancing effect. Moreover, contrary to routine practice, in the present invention, the molecule to be delivered is not encapsulated in any such structure. Rather, the molecules and the liposomal compositions are prepared separately and applied to a region of the skin concurrently or sequentially.

One embodiment of the invention is a method of enhanced delivery of molecules to or through a delivery site on skin in a subject comprising the steps of:

(a) preparing separately: (i) a composition comprising the molecules to be delivered and (ii) a liposomal composition comprising negatively charged lipids;

(b) applying the molecules to be delivered to the delivery site on skin concurrently or sequentially with the liposomal composition;

(c) applying at least one electric pulse to the delivery site of skin concurrently or sequentially with the molecules and liposomal composition of (a), wherein the electric pulse is of sufficient duration and voltage to induce electroporation and delivery of molecules into or through the skin, and wherein the amount of molecule delivered is enhanced if the liposomal composition is applied to the skin.

Another embodiment of the present invention is a method of enhanced delivery of molecules to or through a delivery site on skin in a subject comprising the steps of:

(a) applying the molecules to be delivered to the delivery site on skin concurrently or sequentially with a liposomal composition comprising negatively charged phospholipids;

(b) applying between one and 300 electric pulses to the delivery site of skin concurrently or sequentially with the molecules and liposomal composition of (a), wherein the electric pulse is of a duration of about 10 μsec to about 200 msec and a voltage of about 80 to about 200 V to induce electroporation and delivery of molecules into or through the skin, and wherein the amount of molecules delivered is enhanced when the liposomal composition is applied to the skin as compared to the amount of molecules delivered when the liposomal composition is not applied.

A further method of the invention is a method of increasing the permeability of the SC layer of the skin comprising applying at least one electric pulse to the SC layer of the skin concurrently or sequentially with application of a liposomal composition comprising negatively charged lipids, wherein the permeability of the SC layer as measured by the lifetime of pores formed, is higher than when the liposomal composition is not applied to the skin.

### BRIEF DESCRIPTION OF THE DRAWINGS

FIG. 1 is a representation of enhancement of the relative transport of methylene blue (MB) through heat-stripped porcine stratum corneum by DOPG:DOPC liposomal compositions. The relative transport of MB in the presence (▪) or absence (●) of DOPG:DOPC liposomal compositions is shown after the application of negative pulses for 10, 20 and 30 min and after an additional 10 min without pulse.

FIG. 2 is a representation of the effect of DOPG:DOPC MLV on the transport of MB through porcine SC by electroporation using positive pulses for 10, 20 and 30 min and after an additional 10 min without pulse application. The data were generated in the presence of the lipid formulation (▪); and in the absence of lipid (●).

FIG. 3 is a representation of the relative transport of protoporphyrin IX (PP-IX) through porcine SC by electroporation (negative pulse) in the presence (▪), or absence (●) of DOPG:DOPC liposomal compositions. PP-IX transport was measured after 10, 20 and 30 minutes of pulsing. The last measurement was made 10 minutes following the end of pulsing.

FIG. 4 is a representation of the relative transport of methylated PP-IX (MPP-IX) by electroporation with (▪) and (●) without treatment with DOPG:DOPC MLVs. The porcine SC was pulsed for a total of 30 minutes. The last measurement was made 10 minutes following the end of pulsing.

FIG. 5 is a representation of the transport of FITC-dextrans of varying molecular weights through porcine SC following electroporation in the presence (empty bars) or absence (solid bars) of lipids.

FIG. 6 is a representation of transport of the neutral dextrans Texas-Red Dextran (3 kDa) and Rhodamine-dextrans (10 kDa, 40 kDa and 70 kDa) through porcine SC by electroporation with (empty bars) and without (solid bars) added lipids.

FIG. 7 is a plot of initial SC resistance for increasing numbers of negative pulse application with (●) or without (▪) added lipid formulation.

FIG. 8 is a representation of the percent recovery of SC resistance after the application of 180 pulses of 150V with (●) or without (▪) added lipid formulation.

FIG. 9 is a representation of the percent recovery of SC resistance without added lipids after 60 pulses at 80V (♦), 104V (□), 116V (Δ), 160V (X), 188V (▴) and 300V (●).

FIG. 10 is a representation of the recovery of SC resistance with added lipids after 60 pulses at 80V (♦), 120V (□), 128V (Δ), 156V (X), 188V (▴) and 308V (●)

### DETAILED DESCRIPTION OF THE INVENTION

The present invention provides a method for transdermal or intradermal delivery of molecules through or into skin. The method comprises the steps of providing the molecules to be delivered; providing a liposomal composition, applying the molecules and the liposomal composition concurrently or sequentially to a region of the skin; and applying electrical pulses concurrently or sequentially with application of the molecules and the lipid composition to the region of the skin in contact with the molecule and a liposomal composition. The liposomal composition is comprised of negatively charged lipids, preferably phospholipids. In a preferred embodiment, the phospholipids are DOPG and DOPC, in a ratio of 1:1.

A distinguishing feature of the present invention is that in contrast to methods in the prior art, molecules are delivered without being encapsulated in liposomes. Consequently, the lipid compositions need not be formed into liposomes or other structures to provide the enhancing effect and the molecule to be delivered is not encapsulated in any such structure. Instead, the liposomal components, whether formulated into a structure or not, are used to enhance permeability of the target site for delivery of the molecule.

This discovery that the enhancing effect of liposomal compositions is separate from the molecule delivery function of the liposomes is contrary to the common belief that liposomes encapsulating a molecule are transported intact into the skin and then release the molecule to the target cell or tissue. Without being bound by any particular theory, it appears that the lipid components extend the lifetime of electropores formed during electroporation and it is in this way that they enhance the total transport of molecules after electroporation. Not only can more molecules pass through SC when the pores remain open longer, but a longer pore life also enhances the ability of difficult-to-transport-molecules to pass through SC. The longer pore life also may reduce the total number of electric pulses required to effect sufficient electroporation and delivery.

The enhancement offered by the present invention is generally higher when used in the delivery of neutral molecules, which generally do not electrophorese well. Delivery of charged molecules to and through the SC also is enhanced by the present method when the correct polarity of electric pulse (in relation to the molecule) is used.

Thus, this invention can be used to facilitate the transport of molecules by electroporation, including but not limited to large, neutral molecules that have previously been difficult to transport.

The method comprises the application of electrical pulses concurrently or sequentially with application of the molecules and a lipid composition comprising negatively charged liposomal compositions, which, with respect to each other, can be applied concurrently or sequentially. In one embodiment, the molecule to be delivered can simply be mixed with the liposomal compositions. Simple mixing of the delivery molecules and the liposomal composition is not expected to result in the encapsulation of the molecules in the liposomes. Because encapsulation of molecules is not required, the present method can result in savings in time and cost.

The molecules, the liposomal composition, and the at least one electric pulse are applied to the delivery site on skin in a delivery mode selected from the following:

| | | | Apply | |
|---|---|---|---|---|
| | | | molecule- | Apply at |
| | | Apply | liposomal | least one |
| | Apply | liposomal | composition | electric |
| Delivery | molecule | composition | mixture | pulse to |
| Mode | to skin* | to skin | to skin | skin |
| (a) | 1 | 2 | na | 3 |
| (b) | 1 | 3 | na | 2, 4 |
| (c) | 2 | 3 | na | 1 |
| (d) | 2 | 4 | na | 1, 3, 5 |
| (e) | 4 | 2 | na | 1, 3, 5 |
| (f) | 3 | 2 | na | 1 |
| (g) | 3 | 1 | na | 2, 4 |
| (h) | 2 | 1 | na | 3 |
| (i) | 2 | 3 | na | 1, 4 |
| (j) | 3 | 2 | na | 1, 4 |
| (k) | na | Na | 1 | 2 |
| (l) | na | Na | 2 | 1 |
| (m) | na | Na | 2 | 1, 3 |
| (n) | 1 | 1 | na | 1 |
| (o) | Na | Na | 1 | 1 |
| (p) | 3 | 1 | na | 2 |

*The numbers 1, 2, 3, 4, 5 indicate the first, second, third, fourth and fifth order of sequential events. If the same number appears in every applicable box (e.g., (o)) then the events are concurrent. If different numbers appear in every applicable box (e.g., (c), (d)), then the events are sequential. If more than one number appears in a box, then that event occurs more than once.
“na” means that event is not applicable.

The immediately preceding table is illustrative of the various delivery modes that may be employed. The absence of a delivery mode in the table is not to be interpreted as outside the scope of the present invention. The present invention contemplates the concurrent or sequential application of molecule, liposomal composition, and electric charge in any combinations that will effect electroporation-mediated delivery.

By way of example, the following textual descriptions correspond to several of the delivery modes in the immediately preceding table:

(a) application of the molecules to the skin, followed by application of the liposomal composition to the skin, followed by application of at least one electric pulse to the skin;

(b) application of the molecules to the skin, followed by application of at least one electric pulse to the skin, followed by application of the liposomal composition to the skin, and followed by application of at least one electric pulse to the skin;

(k) mixing the liposomal composition and the molecules together to form a mixture and applying the mixture to the skin. Such mixing does not result in encapsulation of the molecules in the liposomes. This is followed by application of at least one electric pulse to the skin.

The method comprises the application of electrical pulses in a process termed electroporation. Electroporation is considered to involve the formation of pores in the SC layer so that desired molecules may be delivered through the pores intradermally or to the tissue underlying the skin. In the present invention, the delivery of molecules through the skin is enhanced by combining electroporation with exposure of skin to a liposomal composition. By enhanced delivery is meant that the amount of molecule delivered in or through the skin is higher when electroporation is used in combination with exposure of the skin to a liposomal composition, than in the absence of the liposomal composition. The application of electrical pulses, molecule and liposomal composition can occur concurrently or sequentially. For electroporation, negative polarity of pulses generated by any standard apparatus known to those skilled in the art may be used. Generally, at least a positive and a negative electrode are applied to a selected region of the skin. The skin may be shaved, or otherwise removed of hair, if appropriate.

Preferred surface electrodes for use in the invention include, but are not limited to, meander electrodes, micropatch electrode, caliper or other small plate electrodes. Preferred invasive electrodes are microneedle arrays. When invasive electrodes are used, it is preferred that they be minimally invasive.

The liposomal compositions useful for the present invention comprise negatively charged lipids. Suitable examples are dioleoylphosphatidylglycerol (DOPG), phosphatidylserine and diphosphatidylglycerol (cardiolipin). In addition, free fatty acids may also be used since they are negatively charged. The negatively charged lipids may be used alone or in combination with other negatively charged lipids, or with neutral lipids. An example of a neutral lipid useful for the present invention is dioleoylphosphatidyl choline(DOPC). Preparation of liposomes is well known in the art. One way of preparing liposomes can be accomplished by the following steps. Lipid solutions in chloroform are mixed at desired ratio. The solution is then dried under a stream of inert gas (e.g., nitrogen). The dried lipids are placed under vacuum to remove any remaining solvent. A measured amount of buffer is then added to the dry lipids. The lipids can be dispersed in the buffer by vortexing, sonication or by extrusion through filters with micron sized pores. When the liposomal composition comprises lipid components formed into a MLV, the procedure set forth in Example 1 may be followed to form the liposomal composition.

It should be noted that for the present invention, it is not necessary that the molecule be encapsulated in the liposomes or even that liposomes or other structures are formed. Rather, even though the delivery molecules are not encapsulated in the liposomes, the permeability of the SC, with respect to the molecules, is increased when electrical pulses are applied before, during or after exposure of the skin to the liposomal compositions.

The term “liposomal composition”, “lipid composition”, “liposomal components”, or “liposomes” as used herein for the purpose of specification and claims means a composition comprising negatively charged lipids, whether formed into a liposome, particle, vesicle, microsphere, unilamellar or multilamellar lipid vesicle, or not formed into such a structure.

The terms “molecule”, “drug”, “molecule to be delivered”, “agent”, “desired molecules”, “molecules” and similar terms are meant to include drugs (e.g., chemotherapeutic agents), nucleic acids (e.g., polynucleotides), peptides and polypeptides, including antibodies, immunomodulatory agents and other biological response modifiers. The agent to be delivered may offer therapeutic, preventative, cosmetic, prophylactic, gene therapy or other desired effects to the subject in which the treatment is applied.

The term “antibody” as used herein is meant to include intact molecules as well as fragments thereof, such as Fab and F(ab′).sub.2. The term polynucleotides include DNA, cDNA and RNA sequences, as well as natural or synthetic antisense nucleic acids. The term “biological response modifiers” is meant to encompass substances which are involved in modifying the immune response. Examples of immune response modifiers include such compounds as lymphokines. Lymphokines include tumor necrosis factor, interleukins 1, 2, and 3, lymphotoxin, macrophage activating factor, migration inhibition factor, colony stimulating factor, and alpha-interferon, beta-interferon, and gamma-interferon and their subtypes. In addition, agents that are “membrane-acting” agents are also included in the definition of “molecule to be delivered” and like terms. These agents may also be agents that act primarily by damaging the cell membrane. Examples of membrane-acting agents include N-alkylmelamide and para-chloro mercury benzoate.

The term “concurrently” means that two event occur at substantially the same time. The term “sequentially” or “sequential” means that two events occur one after the other, regardless of how long or short the time between events is.

The chemical composition of the agent or molecule to be delivered will dictate the most appropriate time to administer the agent in relation to the administration of the electric pulse. For example, while not wanting to be bound by a particular theory, it is believed that a drug having a low isoelectric point (e.g., neocarcinostatin, IEP=3.78), would likely be more effective if administered post-electroporation in order to avoid electrostatic interaction of the highly charged drug within the field. Further, such drugs as bleomycin, which have a very negative log P, (P being the partition coefficient between octanol and water), are very large in size (MW=1400), and are hydrophilic, diffuse very slowly into a tumor cell and are typically administered prior to or substantially simultaneous with the electric pulse. In addition, certain agents may require modification in order to allow more efficient entry into the cell. For example, an agent such as taxol can be modified to increase solubility in water which would allow more efficient entry into the cell.

For the method of the present invention, the molecule, a liposomal composition and the electric pulses may be applied to a selected region of the skin concurrently or sequentially. The delivery site on skin can be any region appropriate for the subject, molecule to be delivered, and effect sought after delivery. The arm, leg, neck, or other regions are suitable delivery sites. For concurrent application, an electrode with a reservoir may be used. Preferred surface electrodes for use in the invention include but are not limited to meander electrodes, micropatch electrodes, caliper or other small plate electrodes. Preferred invasive electrodes are microneedle arrays. When invasive electrodes are used, it is preferred that they be minimally invasive. The electrodes may be between 0.1 to 10 mm or larger in diameter. An example of a suitable electrode is the Ag/AgCl skin electrode such as those commercially available (IVM Inc., Healdsburg, Calif.).

The liposomal composition comprising the molecule is added to the reservoir of the negative electrode. The negative electrode and the positive electrode are placed on the selected region of the skin at a suitable distance apart. A standard pulse generator (such as AVTECH model AVR-Gl-C-RPCIBl or the BTX Instrument ECM 830 square wave pulse generator) is used to apply an electric potential between the electrodes. Preferably a potential drop of 60-80 Volts across each skin passage under the electrode is used. The pulse length may be 10 μsec to 200 msec. A preferred pulse length is 1 msec. One or more pulses may be applied. A suitable range is from 1 to 180 pulses with the frequency of 1 Hz. A preferred field strength of each pulse is about 0.05 to 5 kV/cm.

To determine the flux and the delivery parameters of individual molecules, the method of the present invention may be carried out in the isolated SC layer. In addition, the level of the molecule may also be monitored in blood to standardize the parameters.

The present invention will be demonstrated by the following examples which are intended to be illustrative and not restrictive.

### EXAMPLE 1

This embodiment demonstrates the transport of both charged and uncharged molecules by the method of the present invention. The transport of three model molecules, Methylene Blue (MB; molecular weight 374Da), protoporphyrin IX (PP-IX; molecular weight 563Da) and methylated protoprophyrin IX (MPP-IX; molecular weight 593Da) was studied in isolated SC.

Stratum corneum layer was obtained from porcine skin by heat treatment as follows. Fresh pieces of porcine skin were wrapped in aluminum foil and placed in a 60° C. water bath for 5 mintues. The SC was gently pulled away from the remaining tissue. The SC can be used directly or stored on glass microscope slides at 4° C. The SC was then used in a Hanson Vertical Diffusion chamber. This simple device is an accepted model system for studying transport through skin by those skilled in the art. This device contains two compartments which are filled with a suitable buffer (10 mM Tris, 100 mM NaCl, 1 mM EDTA at pH 8.0). One of the compartments acts as the donor and the other as the acceptor. The liposomes and the test molecule are added to the donor chamber. The outermost layer of the skin, the SC forms an effective barrier to the transport of biomolecules. The upper chamber was considered as the outer surface of the skin and the lower chamber as the skin directly below the SC. Platinum wires served as electrodes, one was placed in the upper chamber and the other in the lower chamber. Electric pulses were applied across the SC using a pulse generator.

Lipid formulations were prepared as follows. Dioleoylphosphatidylglycerol and dioleoylphosphatidyl choline was mixed at an approximate 1:1 molar ratio and dispersed in buffer by vigorous vortexing resulting in the formation of multilamellar lipid vesicles (MLV). The molecular weights of the molecules tested range from 200 to 600. The model molecules were chosen for their charge and their respective solubility. MB is positively charged and water-soluble. PP-IX has two carboxylic acid groups and at pH 8 it is negatively charged. PP-IX has low solubility. MPP-IX has no charge and is soluble only in the presence of detergents.

The lipid formulation was placed in the upper chamber and pulsed using negative pulses (375V, 1 msec pulse width, 1 Hz pulse repetition frequency) for 10 min. The model molecule as a solution in buffer was then added to the upper chamber. During the next 10 min no pulses were applied. An aliquot of the buffer was removed from the lower (acceptor) chamber. Pulses were next applied for 10, 20 and 30 min and aliquots removed from the lower chamber for analysis at 10, 20 and 30 min to determine delivery of the model molecule. Another aliquot was removed from the lower chamber 10 min after cessation of pulse application. In control studies, the SC was pre-pulsed for 10 min with buffer only (no lipid) and then the model molecule added in the upper chamber and pulsed for three further periods of 10 min each.

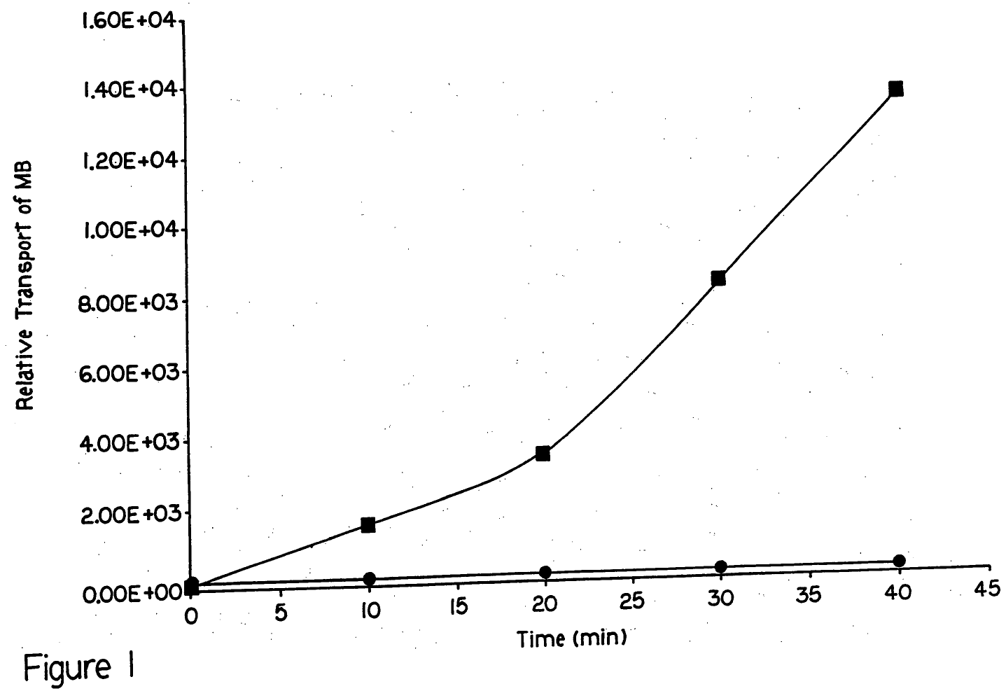

The amount of the model molecule transported to the acceptor chamber was measured in the aliquots removed at different times by using fluorescence spectroscopy. MB, PP-IX and MPP-IX are all fluorescent and this method allows the detection of the model molecules at low concentrations. FIG. 1 shows the time course of transport of MB through porcine skin SC, pre-treated or not pre-treated with the lipid formulation, after electroporation. In the absence of the lipid formulation and even after pulse application for 30 min there is very little MB present in the acceptor chamber. When the SC is pre-treated with the lipid formulation, there is a large amount of MB transported across the SC. Even after cessation of pulse application there is continued increase in MB concentration in the lower chamber indicating diffusion of MB through the SC. Since MB is positively charged and the applied pulses were of negative polarity there could be no electrophoresis of MB through the SC. The results would thus indicate a diffusion of MB through pores created in the SC by the pulse. This diffusion is likely to have occurred in the time between two consecutive pulses.

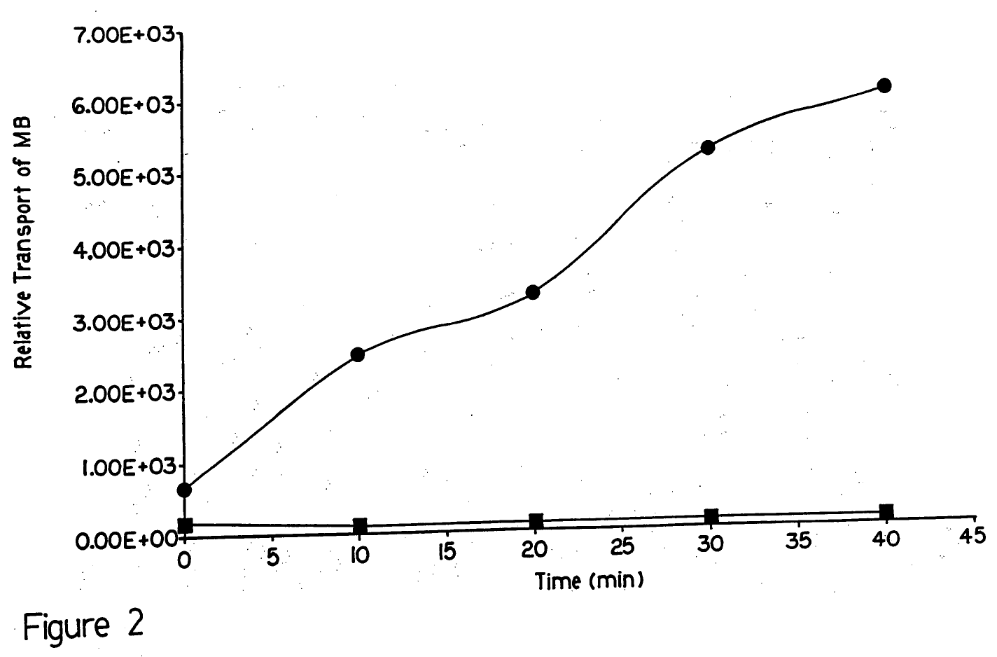

When pulses of positive polarity were applied, the results obtained were exactly opposite of those obtained with negative pulses (FIG. 2). In this case, the lipid formulation inhibited the transport of MB (▪) under conditions suitable for electrophoresis of MB, as apparent from the data obtained in the absence of the lipid formulation (●).

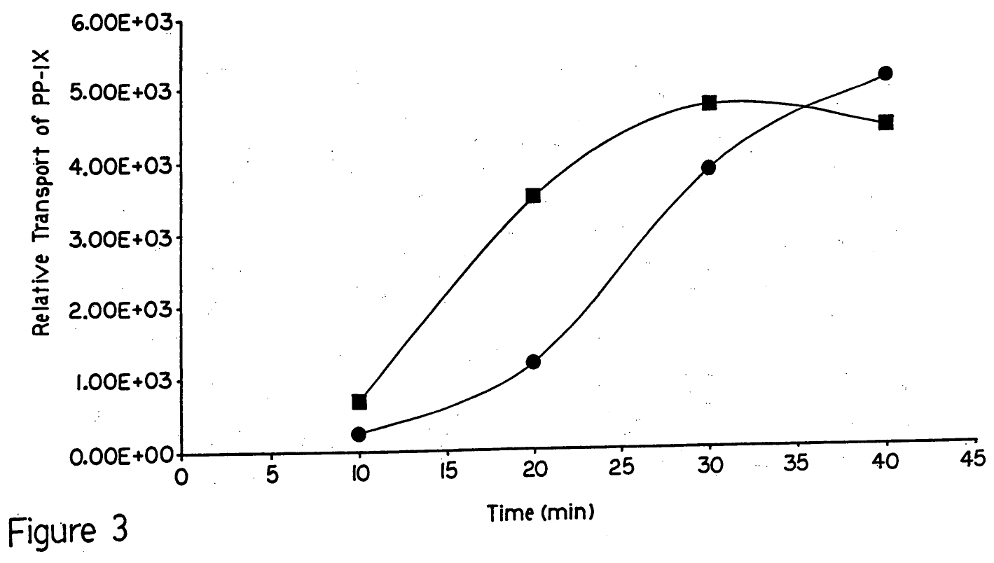

When the negatively charged model molecule PP-IX was used, the results (FIG. 3) show that in the absence of the lipid formulation there is significant transport of PP-IX across the SC. There is however an increase in the transport in the presence of the added lipid. Since PP-IX, due to its charge, will undergo electrophoresis during the pulse, the increase observed in the presence of the lipid is most likely due to diffusion during the time between the pulses. The saturation seen after 30 min is an artifact due to an emptying of the upper chamber of all the solution during 30 min of pulse application.

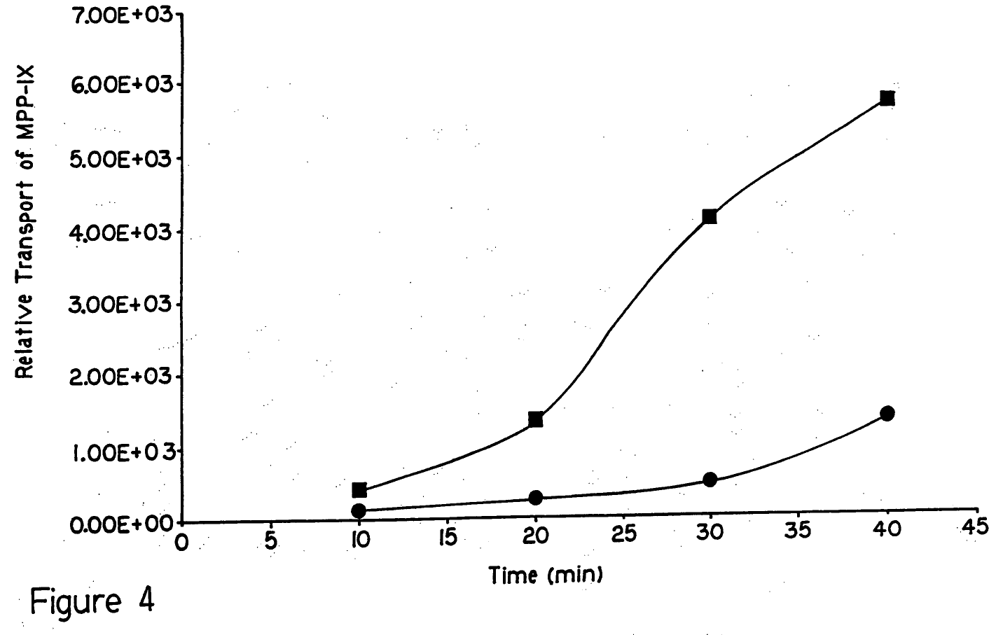

An enhanced transport of the model molecule MPP-IX, an uncharged analogue of PP-IX, is observed when the SC is pre-treated with the lipid formulation and very low transport if the SC was pre-pulsed with buffer alone (no lipid present) (FIG. 4). Since the uncharged MPP-IX will not undergo electrophoresis, the observed transport of MPP-IX is most likely due to diffusion through pores created in the SC by the electropulses. It would thus appear that the diffusion of MPP-IX through such pores is higher when the SC is treated with the lipid formulation.

When similar experiments were carried out with liposomes containing neutral or positively charged lipids, no enhancement of delivery across the SC was observed in the presence of the liposomes.

These data indicate that there is a clear enhancement of transport of molecules when the SC was pre-treated with the lipid formulation and then the model molecule was added. This enhancement is seen for all the model molecules tested irrespective of their charge and water solubility. The increased transport observed when the SC is treated with the lipid formulation can be due to any of the following; (1) increase in the number of electropores, (2) creation of larger pores and (3) pores having longer open lifetime. While not intending to be bound by any particular theory, a possible mechanism of the lipid-induced enhancement could be the incorporation of the negatively charged lipids into the lamellar lipid regions of the SC. The incorporation of these lipids could plausibly increase the fluidity of the SC lipids.

### EXAMPLE 2

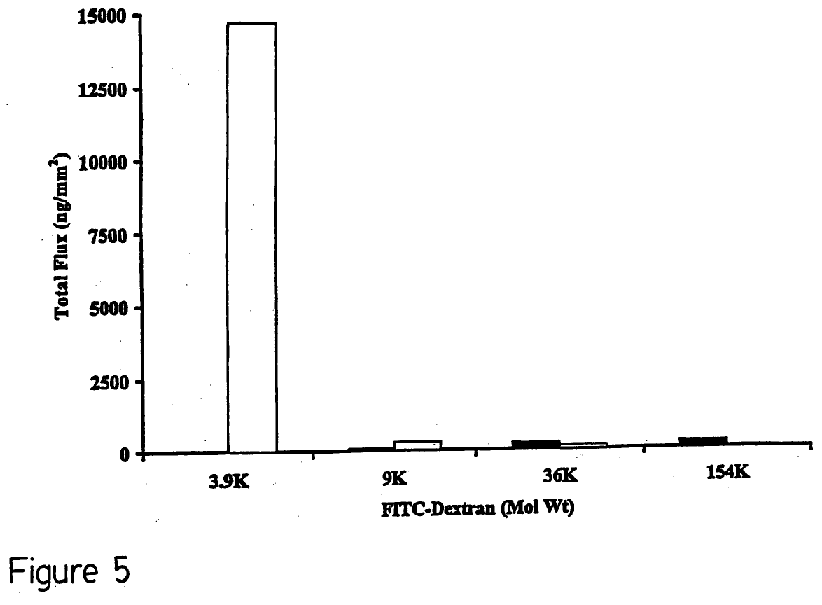

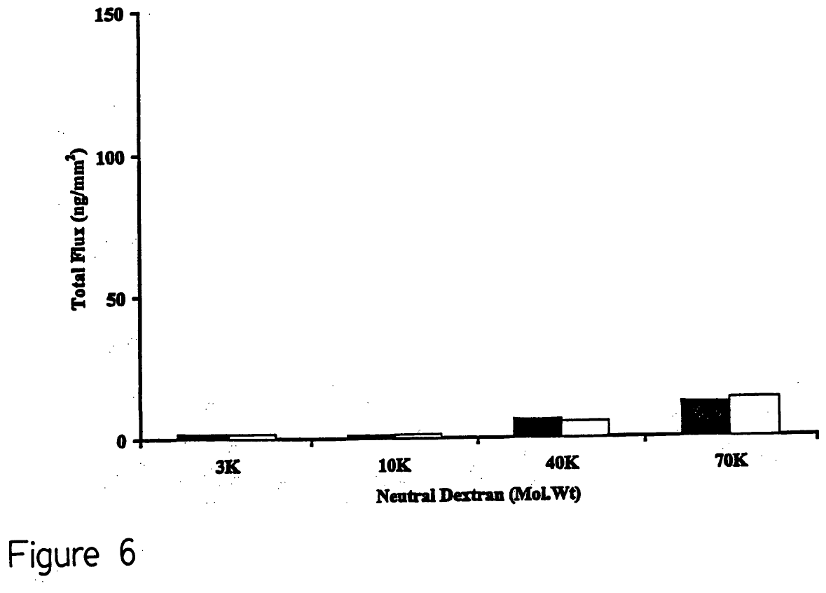

This embodiment demonstrates the effect of lipid formulations on the transport of charged and uncharged dextrans of varying molecular weights. To illustrate this embodiment, the transport of FITC-dextrans of molecular weights 3,900, 9,000, and 154,200 was measured in the Hanson Vertical Diffusion chamber as described in Example 1. Lipid formulation (DOPG:DOPC 1:1, 10 mg/ml) was added to the upper donor chamber along with measured amounts of FITC-dextrans of different molecular weights. Negative pulses, 1 ms duration were applied to the upper (donor) chamber while the lower acceptor chamber was connected to a common ground. After pulse application, the chamber was left undisturbed for 15 min following which the buffer, containing dextrans transported through the SC, was withdrawn from the lower (acceptor) chamber with the help of a syringe. The total buffer was concentrated to 3 ml in a centrifuge vacuum concentrator and the amount of FITC-dextran in the buffer determined by measuring the fluorescence intensity. The measured fluorescence intensity was compared to that obtained using a known amount of FITC-dextrans and measured at identical spectrofluorometric settings. The total flux of FITC-dextrans was calculated based on the cross-sectional area of the SC. As shown in FIGS. 5 and 6, only one of the dextrans, i.e., MW 3,900, had significant transport through porcine SC with added lipids following electroporation. Significant and reproducible transport of larger dextrans (MW>9,000) was not observed under the experimental conditions tested.

### EXAMPLE 3

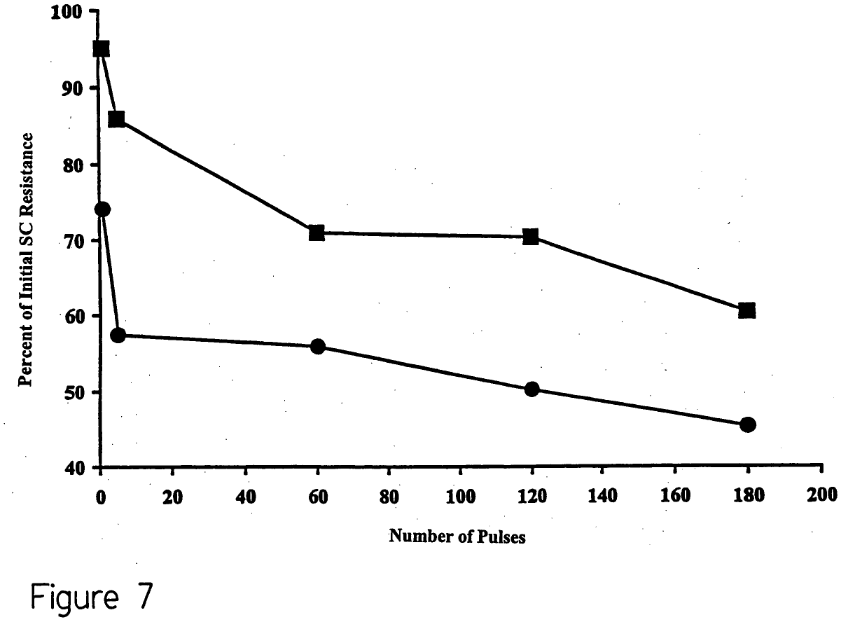

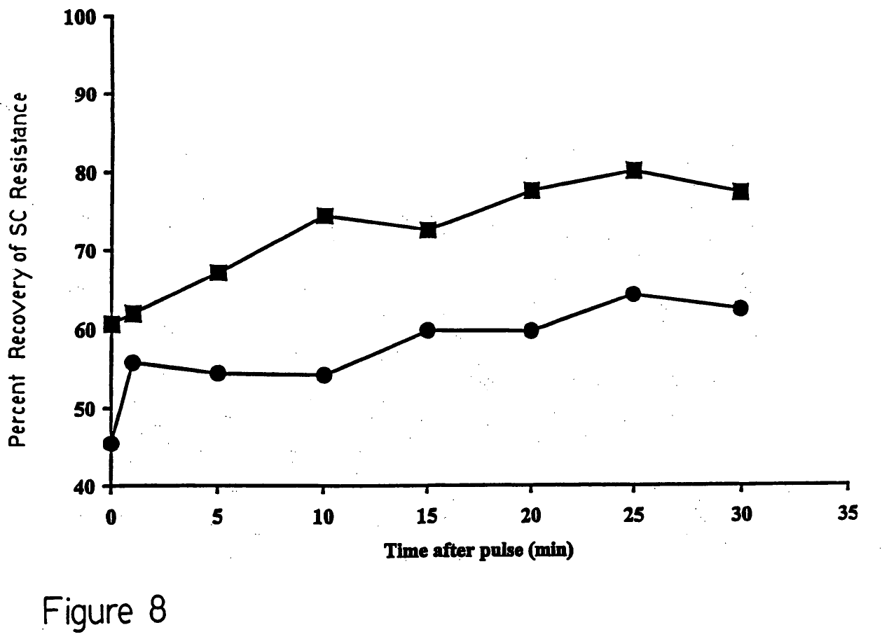

This embodiment demonstrates that the presence of lipid formulation affects the lifetime of pores formed by electroporation in the SC layer of the skin. To illustrate this embodiment, the lifetime of the pores was determined by measuring the recovery of electrical resistance of the SC following electric pulse application. The measurements were carried out using a Hanson Vertical Diffusion chamber as described in Example 2. The resistance of SC was measured using a low voltage AC pulse train. First, the decrease in SC resistance following the application of 1 to 180 pulses of 150 V was measured in the absence and presence of added lipid formulation (DOPG:DOPC 1:1). The results are shown in FIG. 7. The decrease in the SC resistance was greatest after the first few pulses. Subsequent pulses caused only a small additional decrease in the resistance. The decrease in the resistance of the SC in the presence of added lipids was greater than that in the absence of the lipids. After the application of 180 pulses, resistance of the SC was followed for a further 30 min without any further pulse application to determine the rate of recovery of the SC resistance (FIG. 8). The resistance of the SC increased both with and without added lipids. However, the resistance recovery in the presence of added lipid was less than in the absence of lipid. Thus, the SC recovers faster in the absence of added lipids.

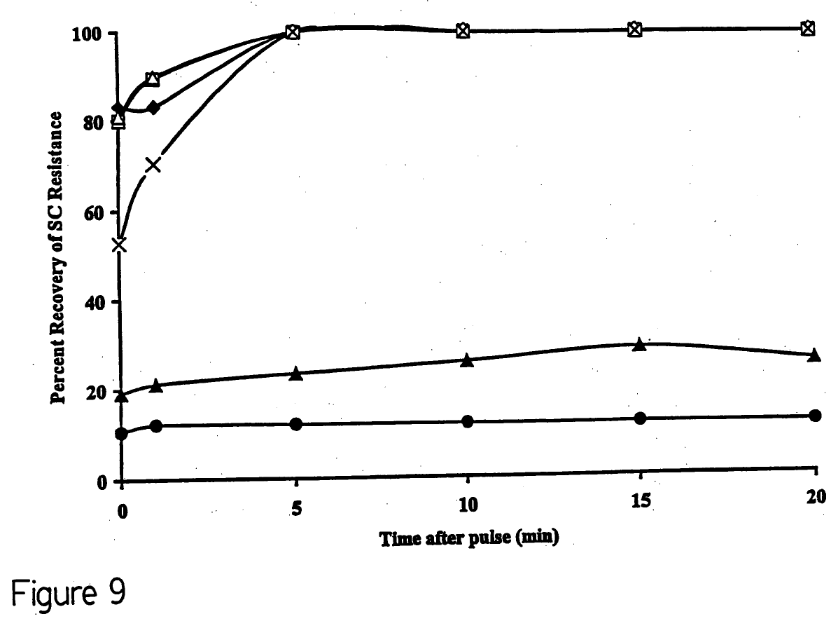

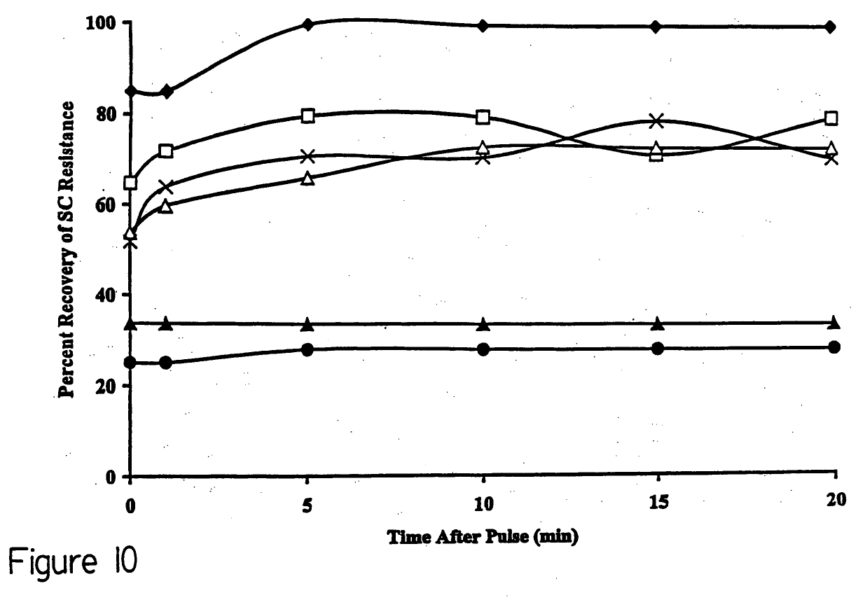

The effect of pulse voltage on the recovery of the SC after pulse application was also determined with and without added lipids. A total of 60 pulses, 1 ms pulse width and was applied at 1 Hz to the porcine SC and the resistance of the SC measured before and after pulse application. The resistance recovery was followed for 20 min after cessation of pulse application. The results are shown in FIGS. 9 and 10 for SC with and without added lipids. There was complete recovery of the SC resistance if the applied pulse voltage was below 200 V in SC without added lipids. Above 200 V there was no measurable recovery of the SC resistance suggesting severe disruption of SC structure. When lipid formulation was added to the SC, there was complete recovery of SC resistance for pulses of up to 80 V. Above 80V, and below 200V there was a partial recovery of SC resistance within the time of the measurements. There was no recovery of SC resistance if the applied pulse voltage was 200 V and higher.

These results indicate that the SC is significantly permeabilized after only a few pulses. The recovery time depends on the pulse voltage and the number of pulses applied. Addition of lipid formulation reduced the total number of pulses required to permeabilize the SC or prolonged the recovery time, respectively.

### EXAMPLE 4

This embodiment describes the method of the present invention in situ. A molecule to be delivered is mixed with a liposomal composition comprising, e.g., DOPG:DOPC 1:1, 10 mg/ml, in a common buffer. The amount of molecule added to the liposomal composition will be determined by the desired concentration of molecule to be delivered. The molecule/lipid mixture is introduced into, e.g., a reservoir in an electrical patch electrode device, having surface-type electrodes. A human subject is prepared by removing the hair from a suitable skin site, such as the arm. The patch electrode is applied to the delivery site and the molecule/lipid mixture is applied to the skin. Single or multiple cycles of electroporation are performed (from about 1 to about 300 pulses), at about 50-100 volts and about 1 Hz, with a pulse length of 1-20 ms. Passive diffusion is allowed between pulsing cycles or between pulses during the cycle and the molecule is delivered.

The data presented herein demonstrate that the method of the present invention can be used for enhanced transdermal delivery of molecules. The foregoing description of the specific embodiments is for the purpose of illustration and is not to be construed as restrictive. From the teachings of the present invention, those skilled in the art will recognize that the devices used and specific conditions mentioned in the present invention may be modified without departing from the spirit of the invention.

What is claimed is:

1. A method of enhanced delivery of molecules to or through a delivery site on skin in a subject comprising the steps of:
(a) separately providing (i) molecules to be delivered and (ii) a liposomal composition comprising negatively charged lipids;
(b) applying the molecules to be delivered to the delivery site on skin concurrently or sequentially with the liposomal composition;
(c) applying at least one electric pulse to the delivery site of skin concurrently or sequentially with the molecules and liposomal composition of (a), wherein the electric pulse is of sufficient duration and voltage to induce electroporation and delivery of molecules into or through the skin, and wherein the amount of molecule delivered is enhanced when the liposomal composition is applied to the skin as compared to when the liposomal composition is not applied to the skin.

2. The method of claim 1, wherein the molecules to be delivered and the liposomal compositions are mixed prior to application to the skin.

3. The method of claim 1, wherein the negatively charged lipids are free fatty acids.

4. The method of claim 1, wherein the negatively charged lipids are phospholipids.

5. The method of claim 4, wherein the phospholipids are selected from the group consisting of dioleoylphosphatidylglycerol (DOPG) and dioleoylphosphatidylcholine (DOPC).

6. The method of claim 1 wherein the at least one electric pulse is applied for a duration of about 10 μsec to about 200 msec.

7. The method of claim 6, wherein the electric pulse is applied for a duration of about 1 msec.

8. The method of claim 1, wherein the field strength of each pulse is about 0.05 to 5 kV/cm.

9. The method of claim 1, wherein the voltage of the electric pulse is about 80 to 200 V.

10. The method of claim 1, wherein the molecule is positively charged, negatively charged or neutral.

11. The method of claim 1, wherein the molecules, the liposomal composition, and the at least one electric pulse are applied to the delivery site on skin in a delivery mode selected from the group of delivery modes consisting of (a) through (p) in the table:

| | | | Apply | |
|---|---|---|---|---|
| | | | molecule- | Apply at |
| | | Apply | liposomal | least one |
| | Apply | liposomal | composition | electric |
| Delivery | molecule | composition | mixture | pulse to |
| Mode | to skin | to skin | to skin | skin |
| (a) | 1 | 2 | Na | 3 |
| (b) | 1 | 3 | Na | 2, 4 |
| (c) | 2 | 3 | Na | 1 |
| (d) | 2 | 4 | Na | 1, 3, 5 |
| (e) | 4 | 2 | Na | 1, 3, 5 |
| (f) | 3 | 2 | Na | 1 |
| (g) | 3 | 1 | Na | 2, 4 |
| (h) | 2 | 1 | Na | 3 |
| (i) | 2 | 3 | Na | 1, 4 |
| (j) | 3 | 2 | Na | 1, 4 |
| (k) | na | na | 1 | 2 |
| (l) | na | na | 2 | 1 |
| (m) | na | na | 2 | 1, 3 |
| (n) | 1 | 1 | Na | 1 |
| (o) | na | na | 1 | 1 |
| (p) | 3 | 1 | na | 2 |

wherein, (i)the numbers 1,2,3,4, 5 indicate the first, second, third, fourth and fifth order of sequential events, (ii) the appearance of the same number in every applicable box indicates concurrent events, (iii) the appearance of a different number(s) in every applicable box indicates the events are sequential, (iv) an event may occur more than once in a delivery mode, and (v) “na” means that event is not applicable.

12. The method of claim 1, wherein the at least one electric pulse is delivered using a surface electrode.

13. The method of claim 12, wherein the surface electrode is selected from the group consisting of meander, micropatch, caliper and small plate electrodes.

14. The method of claim 1, wherein the at least one electric pulse is delivered using an invasive electrode.

15. The method of claim 1, wherein the liposomal composition is a structure selected from the group consisting of liposome, particle, vesicle, microsphere, unilamellar lipid vesicle and multilamellar lipid vesicle.

16. A method of enhanced delivery of molecules to or through a delivery site on skin in a subject comprising the steps of:
(a) applying the molecules to be delivered to the delivery site on skin concurrently or sequentially with a liposomal composition comprising negatively charged phospholipids;
(b) applying between one and 60 electric pulses to the delivery site of skin concurrently or sequentially with the molecules and liposomal composition of (a), wherein the electric pulse is of a duration of about 10 μsec to about 200 msec and a voltage of about 80 to about 200 V to induce electroporation and delivery of molecules into or through the skin, and wherein the amount of molecule delivered is enhanced when the liposomal composition is applied to the skin as compared to when the liposomal composition is not applied to the skin.

17. The method of claim 16, wherein the molecules are positively charged, negatively charged, neutral or combinations thereof.

18. The method of claim 16, wherein the molecules, the liposomal composition, and the at least one electric pulse are applied to the delivery site on skin in a delivery mode selected from the group of delivery modes consisting of (a) through (p) in the table:

| | | | Apply | |
|---|---|---|---|---|
| | | | molecule- | Apply at |
| | | Apply | liposomal | least one |
| | Apply | liposomal | composition | electric |
| Delivery | molecule | composition | mixture | pulse to |
| Mode | to skin | to skin | to skin | skin |
| (a) | 1 | 2 | Na | 3 |
| (b) | 1 | 3 | Na | 2, 4 |
| (c) | 2 | 3 | Na | 1 |
| (d) | 2 | 4 | Na | 1, 3, 5 |
| (e) | 4 | 2 | Na | 1, 3, 5 |
| (f) | 3 | 2 | Na | 1 |
| (g) | 3 | 1 | Na | 2, 4 |
| (h) | 2 | 1 | Na | 3 |
| (i) | 2 | 3 | Na | 1, 4 |
| (j) | 3 | 2 | Na | 1, 4 |
| (k) | na | na | 1 | 2 |
| (l) | na | na | 2 | 1 |
| (m) | na | na | 2 | 1, 3 |
| (n) | 1 | 1 | Na | 1 |
| (o) | na | na | 1 | 1 |
| (p) | 3 | 1 | na | 2 |

wherein, (i)the numbers 1,2,3,4, 5 indicate the first, second, third, fourth and fifth order of sequential events, (ii) the appearance of the same number in every applicable box indicates concurrent events, (iii) the appearance of a different number(s) in every applicable box indicates the events are sequential, (iv) an event may occur more than once in a delivery mode, and (v) “na” means that event is not applicable.

19. The method of claim 16, wherein the at least one electric pulse is delivered using a surface electrode.

20. A method of increasing the permeability of the stratum corneum (SC) layer of the skin comprising applying at least one electric pulse to the SC layer of the skin concurrently or sequentially with application of a liposomal composition comprising negatively charged lipids, wherein the permeability of the SC layer as measured by the lifetime of pores formed, and is higher when the liposomal composition is applied to the skin compared to when the liposomal composition is not applied to the skin.
